(19) United States
(12) Plant Patent Application Publication
(10) Pub. No.: US 2006/0185049 P1
(43) Pub. Date: Aug. 17, 2006

(54) Dianella tasmanica plant named 'DT23'

(76) Inventor: Todd Anthony Layt, New South Wales (AU)

Correspondence Address: MYERS BIGEL SIBLEY & SAJOVEC, PO BOX 37428, RALEIGH, NC, 27627, US

(21) Appl. No.: 11/351,964

(22) Filed: Feb. 10, 2006

Related U.S. Application Data
(60) Provisional application No. 60/652,056, filed on Feb. 11, 2005.

(51) Int. Cl. A01H 5/00

(52) U.S. Cl. PLT/263

(57) ABSTRACT

‘DT23’ is a distinctive variety of Dianella tasmanica, which is characterized by its combination of compact form, green leaf color, broad leaf width, strong leaf arching, green summer basal sheath color with red-brown margin, and large bluish-purple berries.

### RELATED APPLICATIONS

This application claims priority to U.S. Provisional Patent Ser. No. 60/652,056, filed Feb. 11, 2005, the disclosure of which is hereby incorporated by reference in its entirety.

### LATIN NAME OF THE GENUS AND SPECIES

The Latin name of the novel variety disclosed herein is Dianella tasmanica ‘DT23’.

### VARIETY DENOMINATION

The inventive variety of Dianella tasmanica disclosed herein has been given the variety denomination ‘DT23’.

### BACKGROUND OF THE INVENTION

The present invention relates to a new and distinct variety of evergreen perennial Dianella tasmanica, which has been named ‘DT23’. It is anticipated that the plant of this invention will be marketed under the tradename Emerald Arch. Dianella are a genus of ornamental grass-like plants in the Phormiaceae family. In general, Dianella tasmanica has flax-like leaves. Pale blue flowers on stems up to 40 cm high typically develop in spring and early summer and are followed by green berries that become bluish-purple.

Lineage: The cultivar ‘DT23’ was discovered in 1996 in Clarendon, New South Wales, Australia, during a seedling selection of cultivated Dianella tasmanica. The parent (D. tasmanica, ecotype from southern tablelands of new South Wales, Australia) is characterized by green leaf color, medium leaf width, medium leaf arching, absent to very weak leaf glaucosity, and a red-brown basal leaf sheath color. The selection criterion for ‘DT23’ was broad leaf width.

Asexual reproduction: The new variety, Dianella tasmanica ‘DT23’, was first asexually propagated by vegetative division in the state of New South Wales, Australia in September 1997 and has been asexually propagated since that time by division and micropropagation. The distinctive characteristics of cultivar ‘DT23’ have remained stable and true to type through successive cycles of asexual propagation.

### SUMMARY OF THE INVENTION

‘DT23’ is a distinctive variety of Dianella tasmanica, which is characterized by its combination of compact form, green leaf color, broad leaf blade width, strong leaf arching, green summer basal leaf sheath color with red-brown margin and large bluish-purple berries.

### BRIEF DESCRIPTION OF THE DRAWINGS

FIG. 1 shows an exemplary Dianella tasmanica ‘DT23’ plant at approximately two years of age.

FIG. 2 shows an exemplary Dianella tasmanica ‘DT23’ plant at approximately fifteen months of age

### DETAILED BOTANICAL DESCRIPTION OF THE VARIETY

The following is a detailed botanical description of a new and distinct variety of Dianella tasmanica known as ‘DT23’ based upon observations of 15-month-old plants grown in nursery pots in full sun in open beds in Clarendon, New South Wales, Australia. Color notations are based on The Royal Horticultural Society Colour Chart, The Royal Horticultural Society, London, 1995 edition.

Those skilled in the art will appreciate that certain characteristics will vary with older or, conversely, with younger plants. ‘DT23’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions.

‘DT23’ is an evergreen perennial Dianelia tasmanica. ‘DT23’ is a broad-bladed, compact plant, with strong leaf arching, green leaf color, green summer basal leaf sheath color with red-brown margin and large bluish-purple berries.

A botanical description of ‘DT23’ and comparisons with other varieties of Dianella tasmanica are provided below.

Technical Description of the Variety.

Plant characteristics: Growth habit erect to semi-erect, height medium, shoot density medium.
Leaves: Attitude erect to semi-erect, width broad (mean 29 mm), upper side color yellow-green (RHS 147A), lower side color yellow-green (RHS 147B), glaucosity weak, shape ligulate, apex acute, cross-section concave, margin with spines present with medium prominence, midrib lower side with spines present with strong prominence.
Basal sheath: Anthocyanin coloration (summer) red-brown on margin only.
Basal Shoots: Attitude erect to semi-erect, arrangement cluster.
Flowers and berries: ‘DT23’ has been observed for approximately five years and has produced pale blue flowers on stems up to 40 cm high, typically developing in spring and early summer, which are followed by large green berries that become bluish-purple.
Cultural conditions: ‘DT23’ can tolerate low nutrient conditions; it does not like continually wet soil conditions, but can tolerate well-draining sandy soils to very heavy clay soils. ‘DT23’ is fairly adaptable to a wide range of soil pH.
Cold and Heat Tolerance: ‘DT23’ has been observed to be cold tolerant to −12° C. in Clarendon, New South Wales, Australia. Foliage color did not change under these conditions, except for a slight burning at the very tips of the leaves. The plant has also been grown for two years in Charleston, S.C., USA and has remained evergreen down to −6° C.
‘DT23’ is also very heat tolerant. It adapted well to the high heat conditions during summer in Clarendon, New South Wales, Australia and high heat and humidity conditions during summer in Charleston, S.C., USA without any noticeable disease or insect damage.
Drought tolerance: ‘DT23’ has very good drought tolerance. ‘DT23’ survived three months without rainfall under hot conditions in non-irrigated garden beds in summer in Clarendon, Australia. ‘DT23’ has also been grown in non-irrigated beds in Charleston, S.C., USA for two years, and has been observed to go as long as 30 days without rainfall.
Pest Resistance: No known pests.

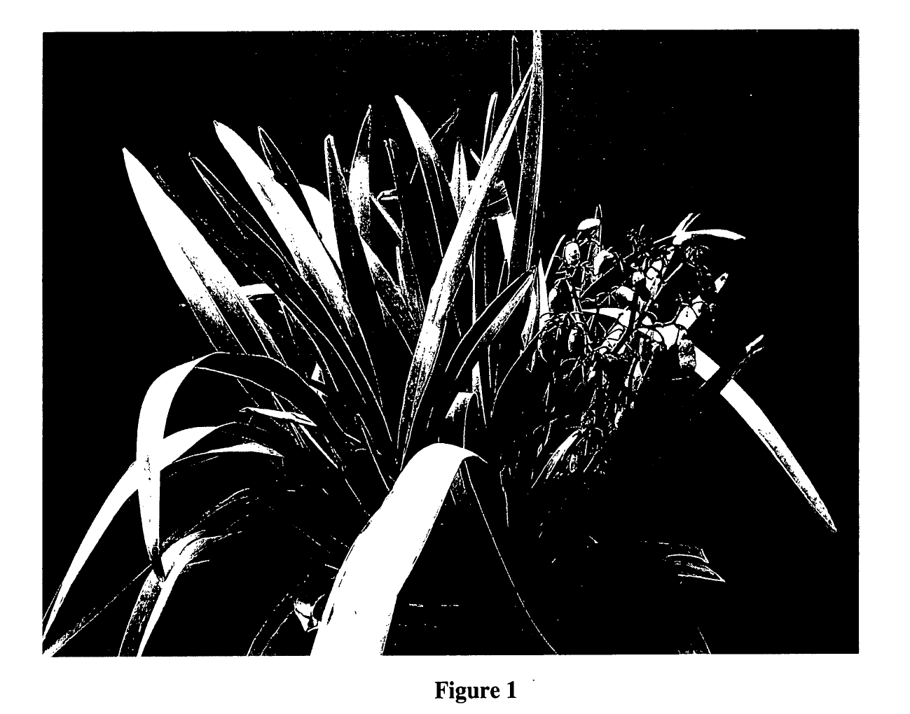

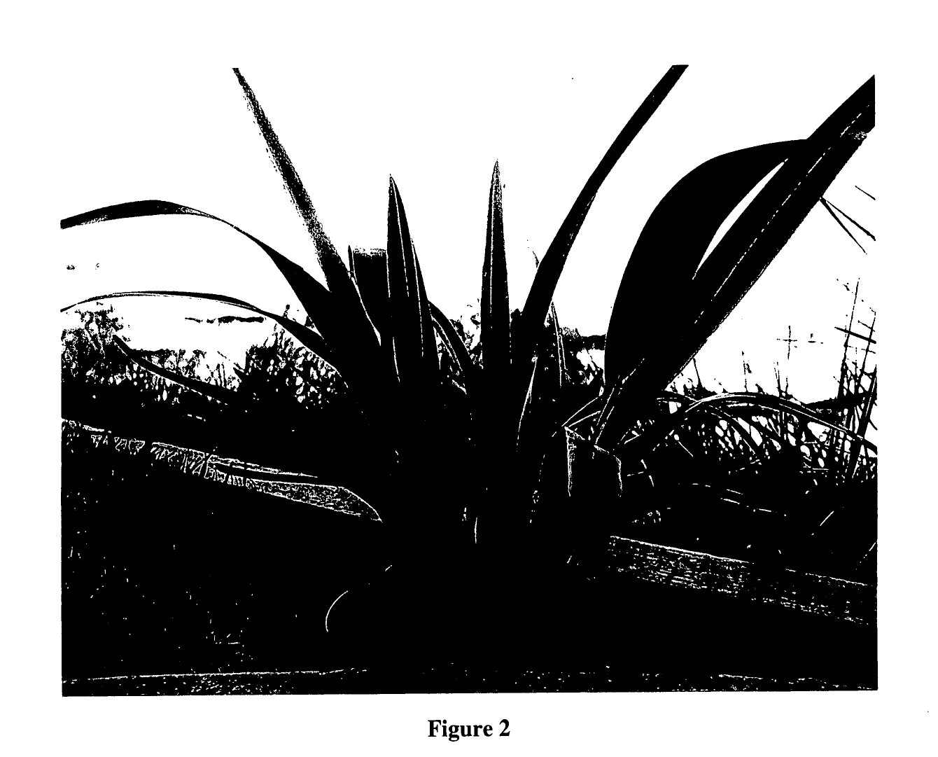

These and other features and characteristics of ‘DT23’ are apparent from FIGS. 1 and 2.

Comparisons with Other Dianella.

‘DT23’ is a more attractive ornamental grass-like plant as compared with the parent. ‘DT23’ is characterized by a yellow-green leaf color, broader leaf blade, stronger leaf arching, and the presence of anthocyanin on the basal sheath margin in summer as compared with the parent ‘D. tasmanica’ (ecotype from southern tablelands of New South Wales, Australia) (see Table 1).

Dianella ‘TR20’ (U.S. patent application Ser. No. 11/238,477, filed Sep. 29, 2005) is the most similar comparator variety to ‘DT23’. In comparison with ‘TR20’, cultivar ‘DT23’ has a green leaf color whereas ‘TR20’ is more yellow-green. ‘DT23’ has a wider leaf, has stronger leaf arching, and more prominent spines on the lower side midrib than ‘TR20’. ‘DT23’ has a green basal sheath color with a red-brown margin in summer whereas ‘TR20’ has an overall red-brown basal sheath color (see Table 1).

A comparative trial of Dianella cultivar ‘DT23’ with ‘TR20’ and the parent D. tasmanica was carried out in summer 2003 to autumn 2004 in Clarendon, New South Wales, Australia. Plant observations and descriptions were taken in autumn 2003. The data are presented in Table 1 below. The plants for this trial were propagated from divisions, planted into 130 mm pots filled with soilless potting mix, and moved into 140 mm pots in full sun for nine months before the trial started. Nutrition was maintained with slow release fertilizers; pest and disease treatments were applied as needed. The trial was designed such that twenty plants of each variety were arranged in a completely randomized manner. Measurements were taken from ten plants at random with one sample per plant.

TABLE 1

| | Dianella Variety | | |
|---|---|---|---|
| Characteristic | ‘DT23’ | ‘TR20’ | D. tasmanica (parent) |
| Plant Height (cm) | | | |
| Mean | 34.2 | 28.0 | 31.5 |
| Std Deviation | 4.5 | 1.9 | 5.7 |
| LSD/Sig. | 4.33 | P ≦ 0.01 | ns* |
| Leaf Width (mm) | | | |
| Mean | 29.1 | 20.0 | 21.3 |
| Std Deviation | 1.6 | 1.7 | 2.0 |
| LSD/Sig. | 1.81 | P ≦ 0.01 | P ≦ 0.01 |
| Leaf Glaucosity | Weak | Absent-very | Absent-very weak |
| | | weak | |
| Leaf lower side | Strong | Medium | Medium |
| midrib: prominence | | | |
| of spines | | | |
| Leaf Color | | | |
| Upper Side | 147A | 146A | 147A |
| Lower Side | 147B | 146B | 147B |
| Basal Sheath Color | Green with | Reddish- | Reddish-brown |
| | red-brown | brown | |
| | margin | | |
| Basal Sheath: | Weak | Medium- | Medium |
| intensity of | | strong | |
| anthocyanin color | | | |

*ns—not significant

That which is claimed is:

1. A new and distinct variety of Dianella tasmanica plant named ‘DT23’, substantially as described and illustrated herein.
